(19) United States
(12) Patent Application Publication
(10) Pub. No.: US 2025/0090273 A1
(43) Pub. Date: Mar. 20, 2025

(54) Systems and Methods for Visualization of Oral Care Treatment Timeline

(71) Applicant: Solventum Intellectual Properties Company, Maplewood, MN (US)

(72) Inventors: Amy L. Unklesbay, Stillwater, MN (US); Cody. J. Olson, Ham Lake, MN (US); Jaymeson M. Tucker, Minneapolis, MN (US)

(21) Appl. No.: 18/728,117

(22) PCT Filed: Feb. 3, 2023
(86) PCT No.: PCT/IB2023/050970

§ 371 Date: Jul. 11, 2024

Related U.S. Application Data
(60) Provisional application No. 63/313,917, filed on Feb. 25, 2022.

(51) Int. Cl. A61C 7/00; G16H 30/00

(52) U.S. Cl. CPC A61C 7/002; G16H 30/00

(57) ABSTRACT

The present disclosure provides a user interface and related methods for displaying the full treatment history of a patient undergoing orthodontic treatment.

### BACKGROUND

Orthodontics is a field of dentistry associated with the professional supervision, guidance and correction of malpositioned teeth. The benefits of orthodontic treatment include attaining and maintaining a proper bite function, enhancing facial aesthetics, and improving dental hygiene. To achieve these goals, the orthodontic professional often makes use of corrective appliances that engage to the patient's teeth and apply gentle therapeutic forces to move the teeth toward proper positions.

One common type of treatment uses tiny, slotted appliances called orthodontic brackets, which are adhesively attached to either the front or back surfaces of the teeth. To begin treatment, a resilient arch-shape wire (“archwire”) is received into the slot of each bracket. The ends of the archwire are generally captured in appliances called molar tubes, which are affixed to the patient's molar teeth. As the archwire slowly returns to its original shape, it acts as a track that guides the movement of teeth toward desired positions. The brackets, tubes, and archwire are collectively known as “braces.”

Orthodontic treatment may also involve the use of alignment trays, such as clear or transparent, polymer-based tooth positioning trays, often referred to as clear tray aligners (CTAs). For example, orthodontic treatment with CTAs may include forming a tray having shells that engage one or more teeth. Each shell may be deformed from an initial position of a tooth, e.g., a malocclusion position. The deformed position of a respective shell of the CTA may apply a force to a respective tooth toward a desired position of the tooth that is an intermediate position between the initial position and a final position resulting from the orthodontic treatment. In some examples, small attachments may be bonded to the teeth to improve force application or achieve desired tooth movements. Proper placement of attachments may ensure proper engagement and interaction of the attachment with a designed feature on the CTAs. The designed feature may provide a desired physical leverage which creates a desired force on a tooth to produce a specific movement of the tooth during treatment. Attachments are typically constructed of varying materials, shapes and sizes, and can be bonded to the labial or lingual surfaces of teeth in order to interact with CTAs and removable appliances in a variety of different ways. Attachments can be applied to a patient's teeth prior to treatment with aligners. Attachments may also be fabricated prior to tooth attachment. Attachments may also be substantially assembled at the orthodontic practitioner's office prior to, or in conjunction with, positioning on the patient's tooth (e.g., molded composites, etc.).

Restorative dentistry may be used to add tooth structure to a patient's dentition, e.g., to an existing tooth, in order to improve at least one of function, integrity, aesthetics, or morphology of missing or irregular tooth structure. For example, restorative dentistry can be an aesthetic treatment to improve appearance of teeth by, for example, altering their shape and/or optical properties (e.g., shade, translucency), which can be achieved using any suitable technique, such as by applying a veneer, managing position or contour of adjacent soft tissues, lessening or removing a gap (diastema) and/or resolving the appearance of malposition. As another example, restorative dentistry may be used to adjust the biting or chewing function of teeth to affect tooth function and/or other aspects of overall oral health such as temporomandibular joint (TMJ) disorders, excessive wear, periodontal involvement, gingival recession or as part of a larger plan to construct a healthy and stable oral environment. Restorative dentistry may be performed in complementary fashion to orthodontic treatment.

### SUMMARY

Like many aspects of twenty first century commerce, dental and orthodontic treatment are increasingly realized over the internet amongst a mix of practitioners and remote entities involved in diagnosis, creation of treatment plans, and fabrication of the tools and appliances necessary to carry out that treatment plan. This holds particularly, but not exclusively, true for the custom orthodontic appliances. A typical orthodontic treatment planning workflow is based on designing an ideal final position of teeth (final setup), then identifying a set of one or more stages used to manufacture or select appliances that will move the teeth from the initial setup to the final setup. For example, a practitioner may make a representation of a patient's dentition (e.g., a digital model or scan, and/or a physical model, which may subsequently be digitized) and send it to a remote site (e.g., a laboratory or appliance manufacturer) via computer or another logistical network. Once created, the digital 3D model then undergoes any necessary preprocessing, which may include data cleanup, tooth segmentation, and tooth coordinate system identification. The digital 3D model may be transmitted along with one or more of: treatment preferences, a number of stages, amount of tooth movement, treatment strategy, and/or an indication of what appliances or combination of appliances (e.g., CTAs and/or brackets) should be used to move the patient's teeth. This data may be used as inputs to generate one or more treatment plans. Each treatment plan may include multiple stages, wherein at each stage of that treatment plan an appliance may be worn for a predetermined period of time; in some variations one or more stage may include orthodontic/dental manipulations (e.g., tooth removal, interproximal reduction, etc.) on the patient's teeth with or without attendant tooth movement. The resulting treatment plan(s) may then be transmitted back to the practitioner for interactive display and selection. The practitioner or other user of the system can optionally make modifications to the treatment plan(s) before: a) sending approval back to the remote site for manufacturing a custom appliance or b) sending approval back to the remote site to aid in pre-existing appliance selection.

Alternatively, any aspect of the treatment plan generation and appliance selection and/or fabrication can occur at the professional's location relying in part on software applications running on a local or remote computer processor. For instance, the treatment plan may include instructions for fabricating CTAs for various treatment stages using additive manufacturing equipment at the practitioner's office. As another option, the practitioner may design a treatment plan locally and subsequently provide instruction to a remote manufacturer.

While patient treatment and tooth movements can be planned prospectively, in many cases orthodontic treatments deviate, both intentionally and unintentionally, from the treatment path prescribed. In some cases, the practitioner may wish to deliberately change the modality or pace of treatment. For example, a practitioner may conclude that a dental arch currently being treated with CTAs would benefit from treatment with braces or ought to be fitted with a retention appliance (e.g., a retainer). In other cases, the deviations arise despite the practitioner's wishes. Such unintentional deviations can arise for numerous reasons, and can include biological variations, poor patient compliance, and/or factors related to biomechanical design, all of which can lead to variability in treatment outcome and decreased treatment efficacy. In any event, the deviations commonly require a change to the current treatment plan. In some cases, these changes occur to the initial or original treatment plan; in others a revised treatment plan is itself subject to further refinement.

A refinement of a patient's treatment plan is typically treated, by either local software or remote manufacturer, as the creation of a new treatment plan. This new, refined treatment plan is commonly displayed as a discreet record without the prior context of the patient's historical treatment. While the patient's historical treatment plans and records remain accessible to the practitioner in other files or views, the refined treatment plan is presented in a virtual vacuum.

The present disclosure provides an integrated treatment timeline display that allows a user to view the entirety of a patient's treatment in a single interface. A single interface reduces the cognitive load of managing multiple individual plans/records and their timing/relation to one another allowing the user to focus on the full expanse of treatment. The single interface further enables related 3D model viewing features to be used across the treatment path. For example, overlays and side-by-side views are no longer limited to use in comparing stages within a single treatment plan, with the user able, using the displays and methods of the present disclosure, to compare stages across the full history of treatment.

In one aspect, the present disclosure provides a computer implemented method for reviewing an orthodontic treatment plan for a patient's dental arch having a current arrangement of teeth, the method comprising the steps of: receiving a refinement request to an original treatment plan including one or more stages of oral care treatment, the refinement including a modification to at least one aspect of the original treatment plan; creating a refined treatment plan, the refined treatment plan including one or more stages of treatment, wherein each stage includes a planned arrangement of teeth in the dental arch and at least one stage includes a) a modified arrangement of teeth as compared to the original treatment plan; b) a different treatment modality; or c) combinations thereof; displaying, via a user interface, a digital representation of at least a portion of the original treatment plan and the refined treatment plan; and providing one or more appliances based on the refined treatment plan.

In the same or other aspects, the request for refinement is received after one or more stages of the original treatment plan have been applied to the patient's dental arch.

In the same or other aspects, displaying the original treatment plan and the refined treatment plan includes editing the original treatment plan to remove any stage that succeeded the request for refinement.

In the same or other aspects, editing the original treatment plan includes receiving an indication of the patient's current arrangement of teeth.

In the same or other aspects, the indication is digital 3D model of the patient's dentition.

In the same or other aspects, the indication is an identification of a particular phase of treatment that best represents the patient's current arrangement of teeth.

In the same or other aspects, the editing step includes syncing the indication with one of the stages of the plurality of stages in the original treatment plan.

In the same or other aspects, creating a refined treatment plan includes changing the arrangement of teeth in at least one stage of the original treatment plan.

In the same or other aspects, creating a refined treatment plan includes changing from a first orthodontic appliance to a second, different orthodontic appliance.

In the same or other aspects, the first orthodontic appliance includes brackets and/or aligners.

In the same or other aspects, displaying a portion of original treatment plan and the refined treatment plan includes displaying a digital 3D representation of a planned arrangement of teeth in the dental arch.

In the same or other aspects, displaying the original treatment plan and the refined treatment plan comprises providing a treatment timeline bar including a representation of each stage of the refined treatment plan and at least one aspect of the original treatment plan.

In the same or other aspects, a digital 3D representation of a planned arrangement of the teeth in the dental arch is provided for each stage of the refined treatment plan.

In the same or other aspects, the digital 3D representation of a planned arrangement is provided for at least each stage of the original treatment plan that occurred prior to the refinement request.

In the same or other aspects, the treatment timeline includes a visible separation between the one or more stages of the original treatment plan and the one or more stages of the refined treatment plan.

In the same or other aspects, stages of the original treatment plan are presented in a different color, shade, or font than the refined treatment plan.

In the same or other aspects, the treatment timeline bar includes a gap between the stages of the original treatment plan and the stages of the refined treatment plan.

In the same or other aspects, the digital representation of at least a portion of the original treatment plan and the refined treatment plan includes model overlays between digital 3D representations of teeth arrangements in a first user selected phase of treatment and a second user selected phase of treatment.

In the same or other aspects, the first user selected phase of treatment and second user selected phase of treatment are both phases of the refined treatment plan.

In the same or other aspects, the first user selected phase of treatment is a phase of the refined treatment plan and second user selected phase of treatment is a phase of the original treatment plan.

In the same or other aspects, the providing step comprises providing one or more aligners and/or brackets.

In the same or other aspects, the providing step comprises providing a combination of aligners and brackets.

In the same or other aspects, the plurality of stages in the revised treatment plan includes one stage of teeth in a final arrangement and at least one stage includes teeth in an intermediate arrangement, the intermediate arrangement representing the teeth in a different arrangement than the current arrangement.

In the same or other aspects, the method further includes providing an original orthodontic treatment plan, wherein the providing includes the steps of: receiving a digital representation of the patient's malocclusion; and creating a target final arrangement of teeth to be achieved at the end of the treatment plan.

In the same or other aspects, the refined treatment plan includes modifying the target final arrangement of teeth.

In the same or other aspects, creating the refined treatment plan includes maintaining the target final arrangement of teeth.

In the same or other aspects, the method further comprises receiving an indication of approval from a practitioner before providing the orthodontic appliances based on the treatment plan.

In another aspect, the present disclosure provides a non-transient, computer-readable medium containing program instructions for causing a computer to: receive, from a remote site, receiving a request after at least one stage of the treatment plan has occurred or the dental arch has reached an arrangement of teeth different than the current arrangement, the revision including a modification to at least one aspect of an original treatment plan; display on a screen, a digital representation of at least a portion of the original treatment plan and a revised treatment plan for user approval, the revised treatment plan including one or more stages of treatment, wherein each stage includes a planned arrangement of teeth in the dental arch and at least one stage includes a modified arrangement of teeth as compared to the original treatment plan; and transmit the revised treatment plan for fabrication of one or more orthodontic appliances after the user has approved the revised treatment plan displayed on the screen.

In the same or other aspects, the program instructions are further configured to create the refined treatment plan.

In the same or other aspects, the program instructions are further configured to display a treatment timeline including a representation of each stage of the refined treatment plan and at least one stage of the original treatment plan.

In the same or other aspects, the digital 3D representation of a planned arrangement is provided for each stage of the refined treatment plan.

In the same or other aspects, the digital 3D representation of a planned arrangement is provided for at least each stage of the original treatment plan that occurred prior to the refinement request.

In the same or other aspects, the treatment timeline includes a visible separation between the stages of the original treatment plan and the stages of the refined treatment plan.

In the same or other aspects, the stages of the original treatment plan are presented in a different color, shade, or font than the refined treatment plan.

In the same or other aspects, the treatment timeline includes a gap between the stages of the original treatment plan and the stages of the refined treatment plan.

In yet another aspect, the present disclosure provides a computer implemented method for reviewing an oral care treatment plan for a patient's dental arch having a current arrangement of teeth, the method comprising the steps of: receiving a refinement request to an original treatment plan including one or more stages of oral care treatment, the refinement including a modification to at least one aspect of the original treatment plan; creating a refined treatment plan, the refined treatment plan including one or more stages of treatment, wherein each stage includes a planned arrangement of teeth in the dental arch and at least one stage includes a) a modified arrangement of teeth as compared to the original treatment plan; b) a different treatment modality; or c) combinations thereof; displaying, via a user interface, a digital representation of at least a portion of the original treatment plan and the refined treatment plan; and transmitting the revised treatment plan for at least one of approval by a practitioner or fabrication of an appliance.

As used herein, “a,” “an,” “the,” “at least one,” and “one or more” are used interchangeably.

As used herein, “oral care” is encompassing of both orthodontic and dental treatments

The above summary of the present disclosure is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exhaustive list.

### BRIEF DESCRIPTION OF THE DRAWINGS

FIG. 1 is a diagram of a system for evaluating oral care treatment plans according to embodiments of the present disclosure;

FIG. 2 is a flowchart for generating a revised treatment plan according to embodiments of the present disclosure;

FIG. 3 is a depiction of a user interface useful in the system of FIG. 1, according to embodiments of the present disclosure;

FIG. 4A-4D are depictions of the user interface of FIG. 3 displaying a full treatment history of a patient, according to embodiments of the present disclosure; and

FIG. 5-6 illustrate example devices that can be used in the embodiments shown in previous Figures.

Features and advantages of the present disclosure will be further understood upon consideration of the detailed description as well as the appended claims. While the above-identified figures set forth several embodiments of the disclosure other embodiments are also contemplated, as noted in the description. In all cases, this disclosure is presented by way of representation and not limitation.

### DETAILED DESCRIPTION

The sections below describe illustrative embodiments directed to an oral care treatment system and methods related thereto. These embodiments are exemplary and accordingly should not be construed to unduly limit the invention. For example, it is to be understood that one of ordinary skill can adapt the disclosed workflows and methods for myriad oral care appliances.

The functions or algorithms described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system, turning such computer system into a specifically programmed machine. The term “processor,” as used herein may refer to any of the foregoing structure or any other structure suitable for implementation of the techniques described herein. In addition, in some aspects, the functionality described herein may be provided within dedicated software modules or hardware modules configured for performing the techniques of this disclosure. Even if implemented in software, the techniques may use hardware such as a processor to execute the software, and a memory to store the software. In any such cases, the computers described herein may define a specific machine that is capable of executing the specific functions described herein. Also, the techniques could be fully implemented in one or more circuits or logic elements, which could also be considered a processor.

Often, computer readable media are provided as part of a computing device. The computing device may have one or more processors, volatile memory (RAM), a device for reading machine-readable media, and input/output devices, such as a display, a keyboard, and a pointing device. Further, a computing device may also include other software, firmware, or combinations thereof, such as an operating system and other application software. A computing device may be, for example, a workstation, a laptop, a tablet, a smart phone, a personal digital assistant (PDA), a server, a mainframe or any other general-purpose or application-specific computing device. A computing device may read executable software instructions from a computer-readable medium (such as a hard drive, a CD-ROM, or a computer memory), or may receive instructions from another source logically connected to computer, such as another networked computer. Data can be communicated directly to an application, e.g., on a mobile device and/or directly to a cloud platform system via cellular connection, a Wi-Fi router or a hub.

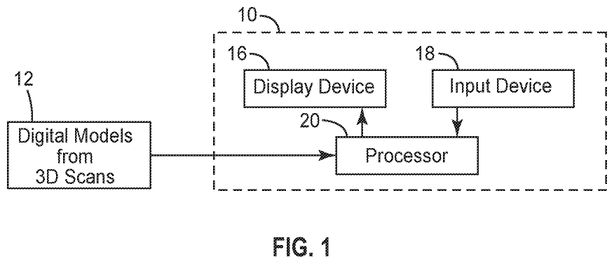

FIG. 1 is a diagram of a system 10 for evaluating oral care treatment plans for orthodontic and dental care (21). System 10 includes a processor 20 receiving digital 3D models of teeth (12) from intra-oral 3D scans or scans of impressions of teeth, or in other embodiments the system receives user manual input. System 10 can also include an electronic display device 16, such as a liquid crystal display (LCD) device, and an input device 18 for receiving user commands or other information. These systems can use an intra-oral scanner to obtain digital images from multiple views of teeth or other intra-oral structures, and those digital images can then be processed to generate a digital 3D model representing the scanned teeth and gingiva. System 10 can be implemented with, for example, a desktop, notebook, or tablet computer. System 10 can receive the 3D scans locally or remotely via a network.

The steps of the process for generating a treatment plan can be implemented as computer program modules for execution on one or more computer systems. Systems and methods for generating a treatment plan can be found, for example, in U.S. Pat. No. 7,435,083 (Chisti et al.), U.S. Pat. No. 7,134,874 (Chisti et al.), U.S. Patent Publication Nos. 2009/0286196 (Wen et al.); 2010/0260405 (Cinader), U.S. Pat. No. 9,259,295 (Christoff et al.) and International Publication No. WO2021/245480 and WO2021/245484 (Cunliffe et al.). The exemplary treatment plan described below is cast specifically to the treatment of a malocclusion with clear tray aligners (CTAs), but steps can be generalized for treatment with labial braces, lingual braces, direct restorations, indirect restorations, and other oral care treatments.

The treatment plan may be stored with other patient information in a patient dental health record (DHR). The DHR may be filled in via information from the patient and/or from treating professionals involved in the patient's care. For example, the DHR can include, but is not limited to including, patient medical information items including x-rays, 3D models of a dental patient's dentition, and/or pictures of the patient's smile. The DHR may also include other medical information, including current and past pharmaceutical prescriptions, health history, genomic information, etc. For patient identifying information, the DHR may include patient name, address, contact information (e.g., telephone number, fax number, electronic mail address), date of birth, gender, and/or dental insurance, among others. The DHR can also include personal treatment goals of the patient (e.g., gap closure, restoration, whitening). At each stage of treatment, the DHR may be updated to reflect treatment progress and include new 3D models of the patient's then-current dentition to aid in diagnosis and further treatment planning.

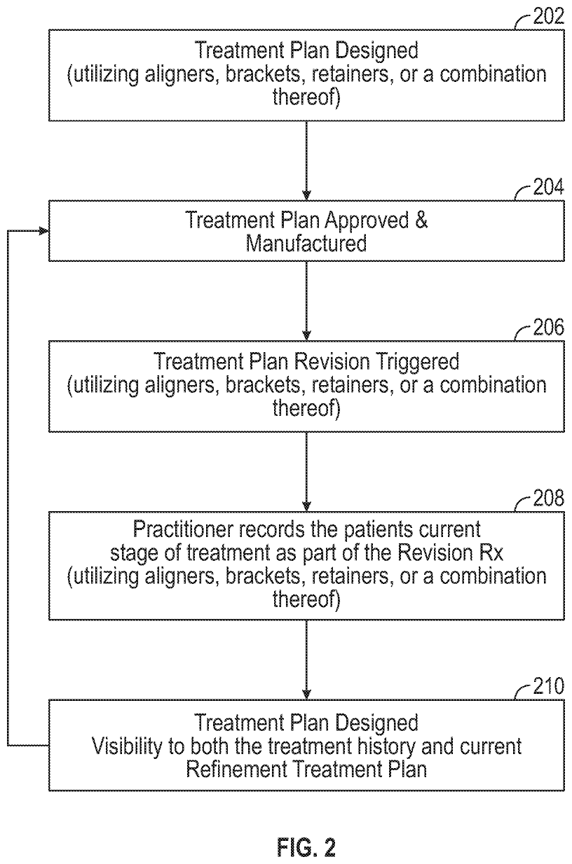

Referring to FIG. 2, a process 200 according for iterative orthodontic treatment is illustrated to convey procedures relevant to the present disclosure. Individual aspects of the process are discussed in further detail below. The process includes receiving information regarding the orthodontic condition of a patient and/or practitioner preferences for treatment and subsequently generating an original treatment plan for repositioning the patient's teeth (Step 202). This original treatment plan may represent the beginning of orthodontic treatment or may represent a new phase of treatment commenced after the patient has undergone some orthodontic or dental treatment. The treatment plan typically includes one or more phases of treatment depending on the desired treatment modality; with CTAs, the treatment plan will include multiple phases of treatment, each corresponding to an arrangement of teeth. For traditional braces, the treatment plan may include a single phase, with a single target arrangement. The treatment plan is presented to a practitioner for modification and/or approval (Step 204). Appliances can be generated or selected based on the approved treatment plan, which will be provided to the practitioner and ultimately administered to the patient.

At some point after approval of the original treatment plan, the practitioner determines that a revision is required. In some embodiments, this determination occurs before the patient wears any appliances due to e.g., errors in the diagnosis of the patient's orthodontic condition, the treatment plan, or the manufacturing of the appliance. In more typical embodiments, this determination occurs after the treatment according to the original plan begins and following administration of appliances to the patient. In such cases, patients do not progress through treatment as expected and deviate from the original treatment plan, whereby an actual tooth arrangement achieved by the patient will differ from the expected or planned tooth arrangement. A determination that the progression of a patient's teeth is deviating or not tracking with the original treatment plan can be accomplished in a variety of ways. Deviations can be detected by visual and/or clinical inspection of the patient's teeth. For example, a deviation from the original treatment plan may become apparent when the patient tries to wear a next appliance in a series and fails to properly seat the appliance given the difference between the actual tooth arrangement and the planned arrangement. In other embodiments, a digital 3D model of the patient's actual tooth arrangement may be compared to the digital 3D model of the planned tooth arrangement.

A user may then request a revision to the treatment plan (Step 206). The revised treatment plan may include the same orthodontic appliance (i.e., same treatment modality) as the original treatment plan. In other embodiments, the revised treatment plan includes a different appliance than the original treatment plan, such as a switch from braces to aligners, or a switch from aligners to a retention appliance, or a switch from restorative dental treatment to orthodontic treatment. The practitioner/user may request for example, to reposition the teeth from the current arrangement to a final arrangement, which may be the same or different destination as the initially determined final arrangement according to the initial treatment plan. Generating the revised treatment plan can include computing new steps of feasible motion of the teeth for only a portion of the initial treatment path and based upon the initial steps and generating the final treatment path with new setups corresponding with the new steps.

The request for revision may be delivered to the remote appliance manufacturer or to treatment planning software running on the user's local processor. The request for revision includes the identification of the current stage of treatment (Step 208). The identification can be a new scan or physical model of the patient's teeth or may be a notation in the request marking the last stage of treatment successfully completed by the patient. The identification of the current arrangement can be used to generate the revised treatment plan (Step 210), as well as govern the presentation of the full treatment history. The revised treatment plan is then displayed to the user, along with at least the stages of the original treatment plan that patient actually experienced.

At various stages, the process for generating the original treatment plan can include interaction with the treating practitioner responsible for the treatment of the patient. Practitioner interaction can be implemented using the network platform and any connected participant. In some or all embodiments, the treatment planning may be embodied within a computer-readable storage medium, such as a computer-readable storage medium of clinician's computing device and/or manufacturer's computer, or both. The computer-readable storage medium stores computer-executable instructions that, when executed, configure a processor to perform the model preparation and treatment planning techniques described below.

Turning to the process steps in more detail, as an initial stage in Step 202, a mold, scan, or photograph of patient's teeth or mouth tissue is acquired. This generally involves direct contact scanning, x-ray imaging, tomographic imaging, sonographic imaging, taking cast, wax bite plates, and other techniques for obtaining information about the position and structure of the teeth, jaws, gums and other relevant tissue. A digital data set is derived from this data that represents an initial (e.g., pretreatment) arrangement of the patient's teeth and other tissues. A computer model of the arch may then be re-constructed based on the data. Alternatively, a practitioner may identify an initial arrangement of teeth by selecting a stage of an existing treatment plan that best represents the patient's malocclusion/current arrangement of teeth, so long as the stage has an associated computer model of the arch.

Desired final positions of the teeth, or tooth positions that are desired and/or intended end result of orthodontic treatment, can be received, e.g., from a treating professional in the form of a descriptive prescription, can be calculated using basic orthodontic prescriptions, or can be extrapolated computationally from a clinical prescription. With a specification of the desired final positions of the teeth and a digital representation of the teeth themselves, the final position and surface geometry of each tooth can be specified to form a complete model of the teeth at the desired end of treatment or treatment stage. The result of this step is a set of digital data structures that represents a desired and/or orthodontically correct repositioning of the modeled teeth relative to presumed-stable tissue. The teeth and surrounding tissue can both be represented as digital data. Further details on software and processes that may be used to derive the target dental arrangement are disclosed, e.g., in U.S. Pat. No. 6,739,870 (Lai et al.), U.S. Pat. Nos. 8,194,067; 7,291,011; 7,354,268; 7,869,983 and 7,726,968 (Raby et al.), and International Publication No. WO2021/245480 and WO2021/245484 (Cunliffe et al.).

Having both a beginning position and a final target position for each tooth, the process can next define a treatment path or tooth path for the motion of each tooth. This can include defining a plurality of planned successive tooth arrangements for moving teeth along a treatment path from an initial arrangement to a selected final arrangement. In one embodiment, the tooth paths are optimized in the aggregate so that the teeth are moved in the most efficient and clinically acceptable fashion to bring the teeth from their initial positions to their desired final positions. A movement pathway for each tooth between a beginning position and a desired final position may be calculated based on a number of parameters, including the total distance of tooth movement, the difficulty in moving the teeth (e.g., based on the surrounding structures, the types and locations of teeth being moved, etc.) and other patient-specific or practitioner-specific data that may be provided. Based on this sort of information, a user or a computer program may generate an appropriate number of intermediary steps (corresponding to a number of treatment steps). In some variations, the user may specify a number of steps, and the software can map different appliance configurations accordingly.

If the movement path requires that the teeth move more than a predetermined amount (e.g., 0.3 mm or less in X or Y translation), then the movement path may be divided up into multiple steps, where each step corresponds to a separate target arrangement. The predetermined amount is generally the amount that an appliance or appliance configuration can move a tooth in a particular direction in the time required for each treatment step. Each appliance configuration corresponds to a planned successive arrangement of the teeth and represents a step along the treatment path for the patient. For example, the steps can be defined and calculated so that each discrete position can follow by straight-line tooth movement or simple rotation from the tooth positions achieved by the preceding discrete step and so that the amount of repositioning required at each step involves an orthodontically optimal amount of force on the patient's dentition. The treatment plan can include a plurality of phases (1 through n) where at time=0, the initial treatment plan begins.

The user/practitioner may be offered several candidate treatment plans for selection as the original treatment plan. The candidate treatment plans can include simulations of treatment using only photographs supplied by the patient, or based on more comprehensive dental imaging (e.g., x-rays, digital scan, etc.) Candidate treatment plans may be generated using a rule-based approach, an optimization-based approach, a machine learning-based approach, or specific preferences (either patient or practitioner) as outlined in WO2021/245484 (Cunliffe et al.). The treatment simulations and representations may further be tied to an estimated cost of treatment. The estimated cost may be based on case difficulty, appliance fabrication cost, patient location, patient treatment goals, number of available treating professionals in the network, estimated completion time, number of treatments requested, or any other factors that tend or may tend to influence ultimate cost to the patient.

The process for generating a treatment plan can also take into consideration interproximal reduction (IPR), which is the removal of some of the outer tooth surface, called enamel. IPR is also known as, and the term IPR includes, slenderizing, stripping, enamel reduction, reproximation, and selective reduction. IPR can be used, for example, between teeth that touch in order to make room to move teeth in orthodontic procedures. The process may apply IPR to a digital 3D model of teeth to simulate application of IPR to actual teeth represented by the model. In other embodiments, a treatment plan may not require consideration of IPR. In other embodiments, the process for generating a treatment plan can model the opposite of IPR using the same or similar techniques, for example as applied to bridges or implants. In this manner, a given stage of the original treatment plan may not represent a different arrangement of teeth as compared to its preceding or succeeding stage, with such a stage instead dedicated to IPR, restorative dentistry, or other oral care treatment.

One or more treatment stages of the treatment plan may include a restorative dentistry treatment. Exemplary tools and methods for dental restoration/restorative dentistry are described in commonly-assigned patent applications United States Patent Publ. No. 2018/0021113, U.S. patent application Ser. No. 16/061,362, entitled “One-Piece Dental Restoration Molds,” filed Dec. 15, 2016, U.S. patent application Ser. No. 16/061,350, entitled “Dental Restoration Molds,” filed Dec. 15, 2016, International Patent Publication Nos. WO 2018/022616 and WO 2020/033532, each entitled “Dental Restoration Molds”, and International App. No. IB2020/054778, filed May 20, 2020 and entitled “Automated Creation of Tooth Restoration Dental Appliances”. Such tools can include a facial mold body for a patient-specific, customized fit with the facial side of at least one tooth of the patient, where the facial mold body includes a restorative portion, at least one aperture aligned with a portion of a surface of a tooth to be restored and at least one door having an inner surface forming a portion of the mold cavity encompassing missing tooth structure of the tooth to be restored. After seating on the arch, the mold cavity can be filled with restorative material (e.g., FILTEK restoratives, available from 3M CompanY) to supply the desired tooth structure.

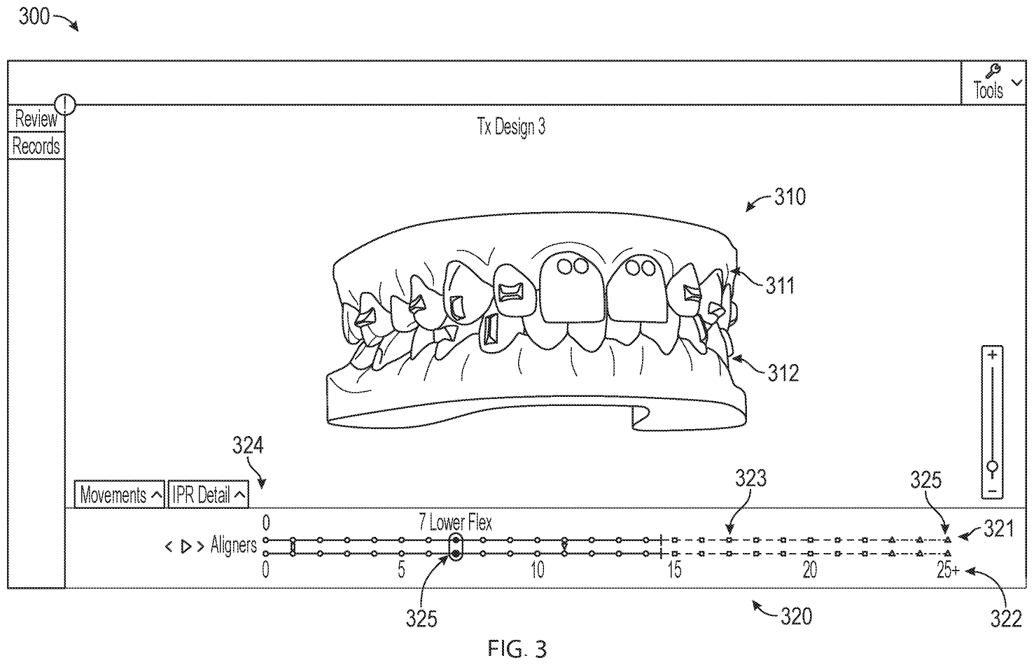

FIG. 3 is a user interface 300 to review and approve an orthodontic treatment plan, displaying a digital 3D model of teeth 310 in each proposed stage of treatment. The user interface may be provided on electronic display device 16. The digital 3D model 310 includes a representation of both of the patient's dental arches 312, 314 in malocclusion, thought the user interface 300 may alternatively present one arch at a time. The user interface 300 can include user interactive tools or commands to zoom in or zoom out, and select a particular view of the digital 3D models of teeth for display (e g., front, top, or side views).

A timeline bar 320 on the bottom of the user interface 300 sequentially arranges the stages of the treatment plan for each dental arch undergoing treatment. The maxillary arch 311 is represented by a first line 321 and the mandibular arch is represented by a second line 322. In some embodiments, only a single line 321, 322 is displayed if a single arch is undergoing treatment. In other embodiments, both timelines 321, 322 will be displayed even if only a single arch is undergoing treatment. Each stage of the treatment plan is represented by a marker 323 disposed on the first and/or second line 321, 322. As depicted, the marker 323 is a circle, but any other shape or form notation can be used to identify a discrete phase. In the depicted embodiment, each colinear, complementary pair of markers 323 represents a specific stage of treatment, including the stage at the beginning of treatment 324 and the target arrangement 325. If, however, only a single arch is undergoing treatment or each arch is treated by a different modality, the markers may not be colinear at each stage. The timeline bar 320 can also include markings to indicate IPR or the bonding of attachments in a particular stage.

A digital 3D model 310 representing the planned arrangement of teeth, in either or both arches, for each stage of treatment is accessible through selecting the pair of markets 323. A user may scroll through the digital 3D models in sequential order or may select a discrete stage of treatment, with a boundary outline 326 identifying the current phase for which the digital 3D model displayed. The user may also be afforded the opportunity to animate the sequence of tooth movement. The user interface 300 can also offer malocclusion overlays, displayed by shading on the digital 3D models of teeth to represent the difference in tooth orientation, position, or geometry between the arrangement of teeth in a given stage of treatment and the current or target arrangement of the teeth. Malocclusion overlays may also be offered between the digital 3D models of two intermediate stages i.e., between the current stage and the target. The user interface 300 may also offer tools to adjust the shading of the malocclusion overlays.

Once the treatment plan is approved, the authorized treatment plan can be sent to a remote appliance manufacturer and used to create or select appliances to effectuate the treatment plan. Alternatively, the treating professional may use the treatment plan to create the necessary appliances in a “chair side” process or select from pre-existing inventory. The resulting appliances may be shipped directly to the patient or provided to practitioner for a clinical seating. The appliances may be provided with a remote monitoring kit to assist the treating professional in tracking the progress of treatment. The remote monitoring kit may include a dental image acquisition support attachable to a patient's mobile phone to assist in the acquisition of dental images. Dental image support devices can be found, for example, in International Publication No. WO2020/089248 (Salah et al.) and as commercially available from GetGRIN, Inc.

After wearing an appliance for a prescribed period of time, the patient may reengage with the practitioner who may evaluate the result of the first iteration of the original treatment plan. In the event that the first iteration of treatment has resulted in satisfactory final occlusion of the patient's teeth, the treatment may be ended. However, if the first iteration of treatment did not complete the desired movement of the patient's teeth, the practitioner may determine whether or not the treatment is still progressing according to plan. To make this determination, the treating professional may take another scan of the patient's teeth or request another remote dental image from the patient's personal computing device to facilitate review. Alternatively, the practitioner may use a bite plate comprising impression material. In presently preferred techniques, at least a portion of the intermediate digital representation is created using a dental imaging device provided to the patient and acquired remote from the practitioner's facility. The practitioner or manufacturer can then utilize these intermediate representations to provide one or more additional appliances that are adapted to provide one or more corrective forces to the teeth such that one or more teeth are repositioned to either a subsequent intermediate arrangement or a final target arrangement. Any suitable technique or combination of techniques can be utilized to provide these intermediate scans, models, and arch members, e.g., the techniques described in U.S. Patent Application Publication No. 2010/0260405 (Cinader) and International Publication WO2016/109660 (Raby et al.).

If a determination is made that the patient's actual arrangement of teeth deviates from a planned arrangement and that the patient's teeth are not progressing as planned, a change or correction in the course of treatment can be selected, for example, by generating a revised treatment plan. In particular, current tooth positions of the patient can be obtained from the patient any one or more phases and compared to models of the patient's teeth according to an earlier or original treatment plan. Where teeth are determined to be deviating from the planned treatment plan, modification or revision of treatment plan can occur. In one embodiment, a revised treatment plan can include restaging the patient's treatment from the actual position to the originally determined final position. The revised treatment path can proceed directly toward the initially determined final position and need not attempt to redirect treatment back onto the original treatment path. Such an approach may be selected, for example, where retaining the initially determined final or target position is desired.

Alternatively, a revised treatment plan can include a more direct “mid-course correction”, in which the revised treatment plan includes a more direct path back toward a planned arrangement of the initial treatment plan. While this approach may make use of the originally planned final arrangement, the more primary concern in this exemplary type of correction is redirecting treatment back to a particular stage of the original treatment plan, rather than from the actual position that is similar but not necessarily exactly the original final position. In yet another embodiment, a revised treatment plan can include essentially “re-starting” treatment and generating a new final arrangement of the teeth and directing the patient's teeth from the actual arrangement to the newly determined final arrangement of the teeth.

As yet another alternative, the revised treatment plan can incorporate a new modality of treatment, meaning the type of appliance used to treat a given arch may be changed by the practitioner. For example, a practitioner may determine that an original treatment plan including CTAs is insufficient or ineffective and may wish to prescribe braces to reach the desired final arrangement of teeth. The practitioner could also determine that a case at or near completion ought to be finished in CTAs. As another example, a practitioner may wish to pause treatment on or both arches and prescribe a retention appliance. As yet another example, an original treatment plan may include restorative dental treatments on one or both arches, and the revised treatment plan may include prescribing one or more orthodontic appliances at or before the conclusion of restorative treatment. One of skill in the art will appreciate the considerations made when switching between treatment modalities, including as set out in, for example, U.S. Pat. No. 7,074,038 (Miller) and US 2015/1032707 (Huang et al.). Furthermore, the practitioner or patient may even seek a revised treatment plan before any appliance is worn or treatment under the original treatment plan commenced.

The revised treatment plan may be stored in the patient's DHR, along with at least a portion of the original treatment plan. In some embodiments, the DHR is edited to account for actual treatment progress. As an example, an original treatment plan may include 10 phases of CTA treatment, each phase including a specific, planned arrangement of teeth. If the patient progresses through only the first 5 phases before a practitioner determines a revision is needed, the remaining 5 phase may be characterized or labeled as non-occurring and/or removed. To aid in the characterization, the practitioner may identify or label the last stage of treatment experienced by the patient in the DHR or revision request, or the manufacturer may identify the last phase by matching a new digital 3D model of the patient's actual arrangement with one of the plurality of planned arrangements.

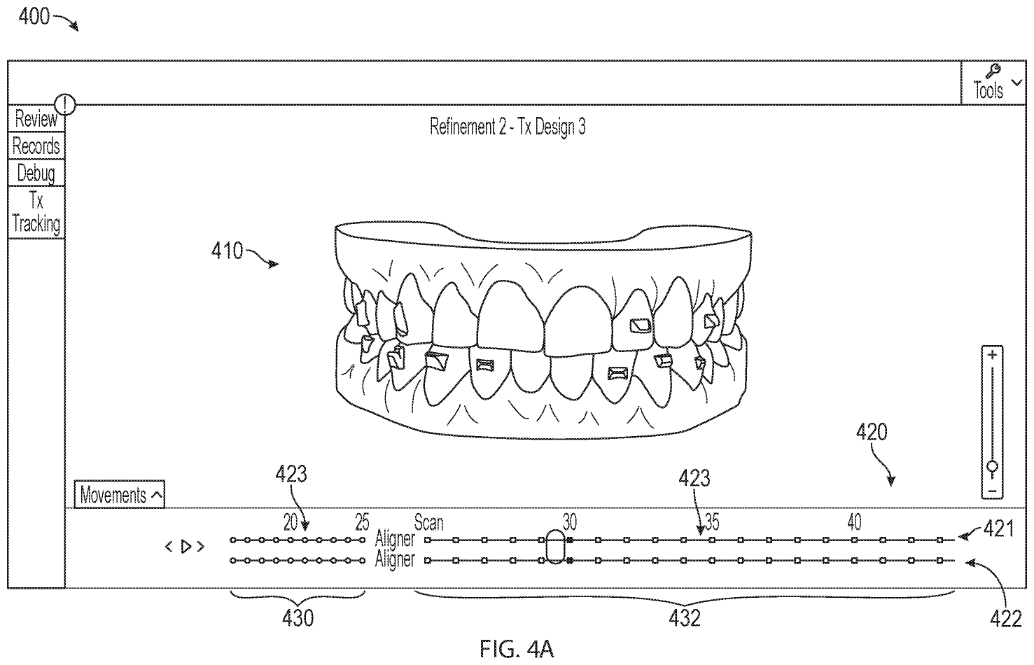

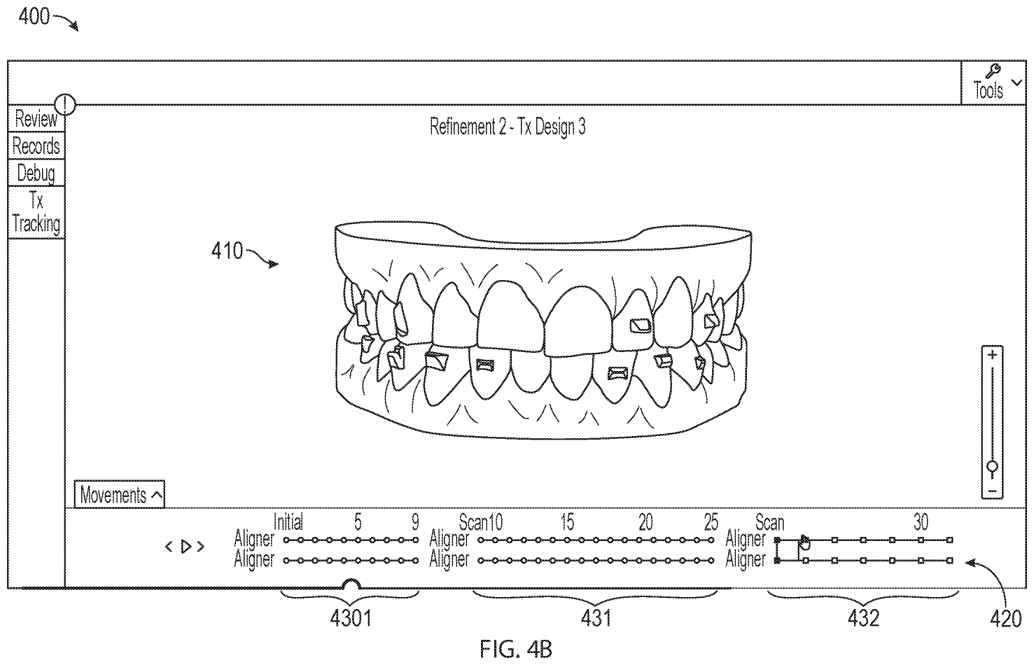

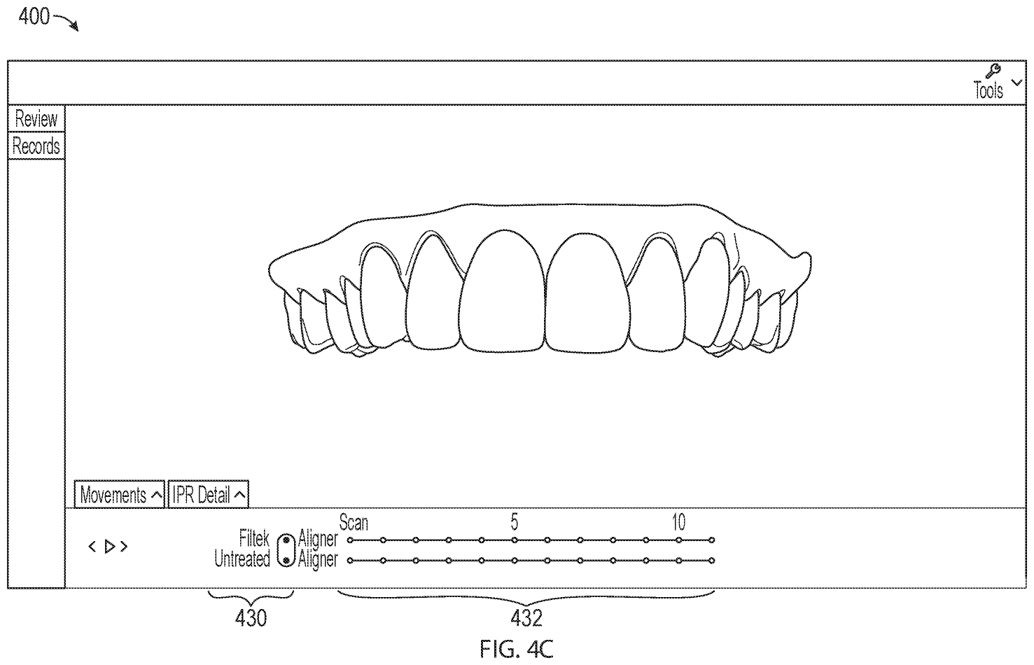

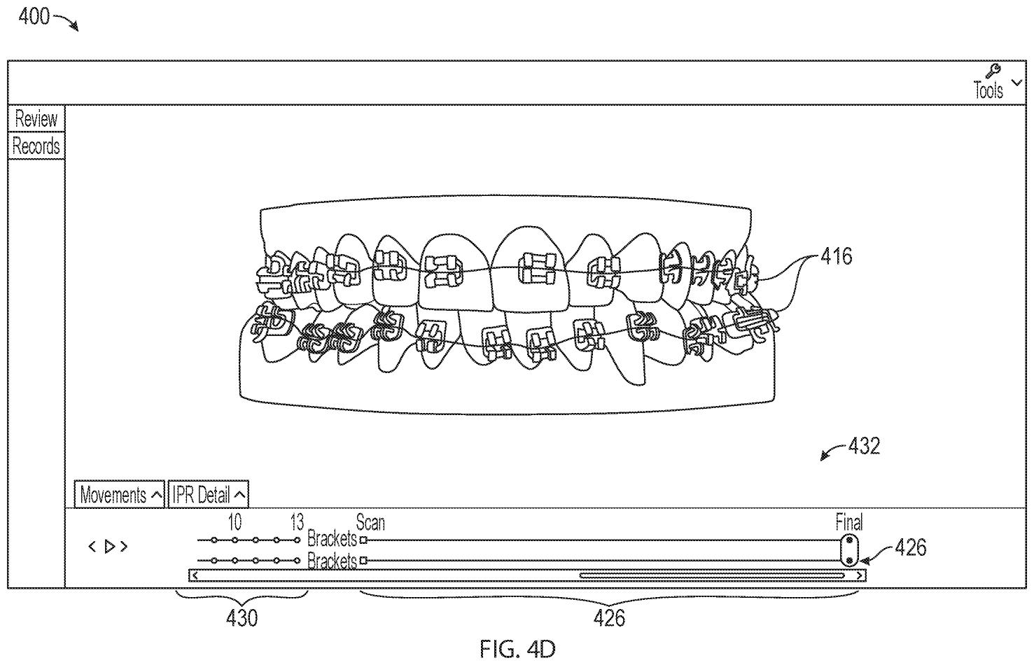

A revised treatment plan may then be generated and provided for approval using the techniques referenced above. FIG. 4A is a user interface 400 to review and approve a revised orthodontic treatment plan, displaying a digital 3D model of teeth 410 in each proposed stage of treatment, as well as display prior stages of treatment represented in the original treatment plan. The user interface 400 may feature the same interactive tools, digital 3D model views, and commands as user interface 300. Like elements are marked with like reference characters, and the skilled person should understand the applicability of such features throughout the following embodiments.

A timeline bar 420 on the bottom of the user interface 400 sequentially arranges, from left to right, the stages of both the revised and original treatment plan for each dental arch undergoing treatment, thus presenting the full record of treatment. The maxillary arch 411 is represented by a first line 421 and the mandibular arch is represented by a second line 422. Each stage of the treatment plan is represented by a marker 423 disposed on the first and/or second line 421, 422. The timeline bar 420 includes a user-perceptible separation between the phases of the original treatment plan 430 and the revised treatment plan 432. The separation may be marked with the type of appliance, as shown, or other signifier of a change in the treatment path. As described above, the original treatment plan 430 may be presented in full or edited to remove any non-occurring stage. The timeline bar 420, markers 423, and other treatment information in the original treatment plan 430 may be presented in a different shade, color, or font than the revised treatment plan 432, but this may not be strictly necessary with the visual separation.

A digital 3D model 410 representing the planned arrangement of teeth for each stage of both the original and revised treatment plans 430, 432 is accessible through selecting the pair of markers 423 (or single marker, if e.g., treatment is restricted to a single arch). A user may scroll through the digital 3D models in sequential order or may select a discrete stage of treatment from either the original or revised treatment plan 430, 432. The user may also be afforded the opportunity to animate the sequence of tooth movement through both the original and revised treatment plans, 430, 432. The user interface 400 can also offer malocclusion overlays, displayed by shading on the digital 3D models of teeth to represent the difference in tooth orientation, position, or geometry between the arrangement of teeth in a given stage of treatment and the current or target arrangement of the teeth. Malocclusion overlays may also be offered between the digital 3D models of two intermediate stages i.e., between the current stage and the target.

FIG. 4B is another depiction of user interface 400 where the current, revised treatment plan 432 is not the first revision in the patient's treatment history. Here, the timeline bar 420 includes a first revision 431 succeeding the original treatment plan 430 but preceding the current revision 432. As described above, the original treatment plan 430 and/or the first revised treatment plan 431 may be presented in full or edited to remove any non-occurring stage.

FIG. 4C is another depiction of user interface 400 where the original treatment plan 430 includes a single stage of restorative dentistry, followed by a revised treatment plan featuring CTAs 432. FIG. 4D is another depiction of user interface 400 where the original treatment plan includes a CTA based, phased treatment plan 430, followed by a revised treatment plan 432 featuring braces 416 on both arches. The revised treatment plan 432 is depicted as only including an initial and final phase, but treatment with braces may include intermediate phases to e.g., change the material of the archwire or bond brackets in new locations.

Different user groups could electronically access the user interfaces 300 and 400, including technicians, doctors, and patients. For technician and doctor use, this tool could be used to modify or aid in the design of the final treatment setup. For patient use, it could be used as a communication tool for the doctor to convince the patient to undergo treatment, and to set realistic expectations for treatment duration and anticipated success at various stages of the treatment. It could also be used to compare different courses of treatment. It could also be used to compare treatments with different modalities, for example clear tray aligners versus brackets and wires, and how each affects the results the patient will see and feel on a regular basis.

The full case presentation through a single user interface 430 reduces the cognitive load of managing multiple individual plans/records and their timing/relation to one another, allowing the user to focus on the treatment in a holistic manner. The single interface further enables related digital 3D model viewing features to be used across the treatment path. This enhanced functionality allows for improved treatment tracking and comparison between the proposed revision and the treatment as actually experienced by the patient, ideally leading to improved insights and treatment outcomes.

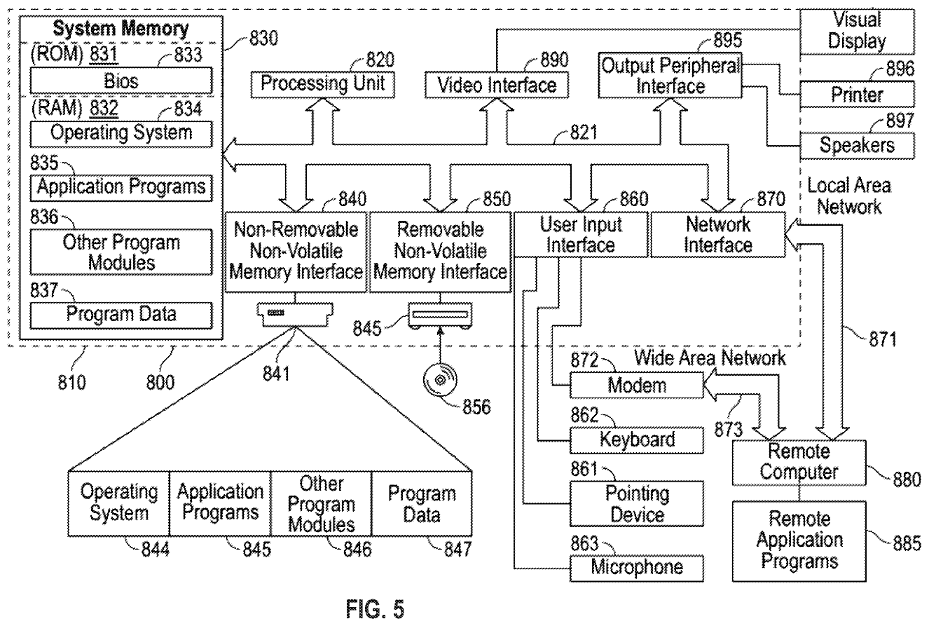

FIG. 5 is one example of a computing environment in which elements of systems and methods described herein, or parts of them (for example), can be deployed. With reference to FIG. 5, an example system for implementing some embodiments includes a general-purpose computing device in the form of a computer 810. Components of computer 810 may include, but are not limited to, a processing unit 820 (which can comprise a processor), a system memory 830, and a system bus 821 that couples various system components including the system memory to the processing unit 820. The system bus 821 may be any of several types of bus structures including a memory bus or memory controller, a peripheral bus, and a local bus using any of a variety of bus architectures. Memory and programs described with respect to systems and methods described herein can be deployed in corresponding portions of FIG. 5.

Computer 810 typically includes a variety of computer readable media. Computer readable media can be any available media that can be accessed by computer 810 and includes both volatile/nonvolatile media and removable/non-removable media. By way of example, and not limitation, computer readable media may comprise computer storage media and communication media. Computer storage media is different from, and does not include, a modulated data signal or carrier wave. It includes hardware storage media including both volatile/nonvolatile and removable/non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 810. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 830 includes computer storage media in the form of volatile and/or nonvolatile memory such as read only memory (ROM) 831 and random-access memory (RAM) 832. A basic input/output system 833 (BIOS) containing the basic routines that help to transfer information between elements within computer 810, such as during start-up, is typically stored in ROM 831. RAM 832 typically contains data and/or program modules that are immediately accessible to and/or presently being operated on by processing unit 820. By way of example, and not limitation, FIG. 5 illustrates operating system 834, application programs 835, other program modules 836, and program data 837.

The computer 810 may also include other removable/non-removable and volatile/nonvolatile computer storage media. By way of example only, FIG. 5 illustrates a hard disk drive 841 that reads from or writes to non-removable, nonvolatile magnetic media, nonvolatile magnetic disk 852, an optical disk drive 855, and nonvolatile optical disk 856. The hard disk drive 841 is typically connected to the system bus 821 through a non-removable memory interface such as interface 840, and optical disk drive 855 are typically connected to the system bus 821 by a removable memory interface, such as interface 850.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc.

The drives and their associated computer storage media discussed above and illustrated in FIG. 5, provide storage of computer readable instructions, data structures, program modules and other data for the computer 810. In FIG. 5, for example, hard disk drive 841 is illustrated as storing operating system 1844, application programs 845, other program modules 846, and program data 847. Note that these components can either be the same as or different from operating system 834, application programs 835, other program modules 836, and program data 837.

A user may enter commands and information into the computer 810 through input devices such as a keyboard 862, a microphone 863, and a pointing device 861, such as a mouse, trackball or touch pad. Other input devices (not shown) may include a joystick, game pad, satellite receiver, scanner, or the like. These and other input devices are often connected to the processing unit 820 through a user input interface 860 that is coupled to the system bus but may be connected by other interface and bus structures. A visual display 891 or other type of display device is also connected to the system bus 821 via an interface, such as a video interface 890. In addition to the monitor, computers may also include other peripheral output devices such as speakers 897 and printer 896, which may be connected through an output peripheral interface 895.

The computer 810 is operated in a networked environment using logical connections, such as a Local Area Network (LAN) or Wide Area Network (WAN) to one or more remote computers, such as a remote computer 880. When used in a LAN networking environment, the computer 810 is connected to the LAN 871 through a network interface or adapter 870. When used in a WAN networking environment, the computer 810 typically includes a modem 872 or other means for establishing communications over the WAN 873, such as the Internet. In a networked environment, program modules may be stored in a remote memory storage device. FIG. 5 illustrates, for example, that remote application programs 885 can reside on remote computer 880.

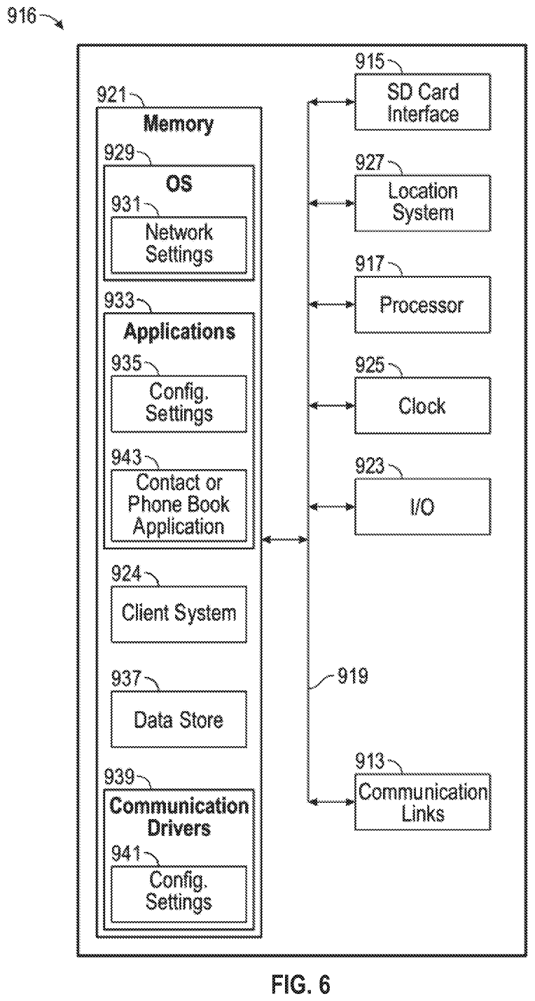

FIG. 6 illustrates an example mobile device that can be used in the embodiments shown in previous Figures. FIG. 6 is a simplified block diagram of one illustrative example of a handheld or mobile computing device that can be used as a user's or practitioner's handheld device, for example, in which the present system (or parts of it) can be deployed. For instance, a mobile device can be deployed in the operator compartment of computing device for use in generating, processing, or displaying the DHR, Tier Subscription, or Treatment Plan.

FIG. 6 provides a general block diagram of the components of a mobile cellular device 916 that can run some components shown and described herein. Mobile cellular device 916 interacts with them or runs some and interacts with some. In the device 916, a communications link 913 is provided that allows the handheld device to communicate with other computing devices and under some embodiments provides a channel for receiving information automatically, such as by scanning. Examples of communications link 913 include allowing communication though one or more communication protocols, such as wireless services used to provide cellular access to a network, as well as protocols that provide local wireless connections to networks.

In other examples, applications can be received on a removable Secure Digital (SD) card that is connected to an interface 915. Interface 915 and communication links 913 communicate with a processor 917 (which can also embody a processor) along a bus 919 that is also connected to memory 921 and input/output (I/O) components 923, as well as clock 925 and location system 927.

I/O components 923, in one embodiment, are provided to facilitate input and output operations and the device 916 can include input components such as buttons, touch sensors, optical sensors, microphones, touch screens, proximity sensors, accelerometers, orientation sensors and output components such as a display device, a speaker, and or a printer port. Other I/O components 923 can be used as well.

Clock 925 illustratively comprises a real time clock component that outputs a time and date. It can also provide timing functions for processor 917.

Illustratively, location system 927 includes a component that outputs a current geographical location of device 916. This can include, for instance, a global positioning system (GPS) receiver, a LORAN system, a dead reckoning system, a cellular triangulation system, or other positioning system. It can also include, for example, mapping software or navigation software that generates desired maps, navigation routes and other geographic functions.

Memory 921 stores operating system 929, network settings 931, applications 933, application configuration settings 935, data store 937, communication drivers 939, and communication configuration settings 941. Memory 921 can include all types of tangible volatile and non-volatile computer-readable memory devices. It can also include computer storage media (described below). Memory 921 stores computer readable instructions that, when executed by processor 917, cause the processor to perform computer-implemented steps or functions according to the instructions. Processor 917 can be activated by other components to facilitate their functionality as well.

All of the patents and patent applications mentioned above are hereby expressly incorporated into the present description. The foregoing invention has been described in some detail by way of illustration and example for purposes of clarity and understanding. However, various alternatives, modifications, and equivalents may be used and the above description should not be taken as limiting in the scope of the invention which is defined by the following claims and their equivalents.

1. A computer implemented method for reviewing an orthodontic treatment plan for a patient's dental arch having a current arrangement of teeth, the method comprising the steps of:
receiving a refinement request to an original treatment plan including one or more stages of oral care treatment, the refinement including a modification to at least one aspect of the original treatment plan;
creating a refined treatment plan, the refined treatment plan including one or more stages of treatment, wherein each stage includes a planned arrangement of teeth in the dental arch and at least one stage includes a) a modified arrangement of teeth as compared to the original treatment plan; b) a different treatment modality; or c) combinations thereof;
displaying, via a user interface, a digital representation of at least a portion of the original treatment plan and the refined treatment plan, wherein displaying the original treatment plan and the refined treatment plan includes editing the original treatment plan to remove any stage that succeeded the request for refinement; and
providing one or more appliances based on the refined treatment plan.

2. The method of claim 1, wherein the request for refinement is received after one or more stages of the original treatment plan have been applied to the patient's dental arch.

3. (canceled)

4. The method of claim 1, wherein editing the original treatment plan includes receiving an indication of the patient's current arrangement of teeth.

5. The method of claim 4, wherein the indication is a digital 3D model of the patient's dentition.

6. The method of claim 4, wherein the indication is an identification of a particular phase of treatment that best represents the patient's current arrangement of teeth.

7. The method of claim 4, wherein editing step includes syncing the indication with one of the stages of the plurality of stages in the original treatment plan.

8. The method of claim 1, wherein creating a refined treatment plan includes changing from a first orthodontic appliance to a second, different orthodontic appliance.

9. The method of claim 1, wherein displaying the original treatment plan and the refined treatment plan comprises providing a treatment timeline bar including a representation of each stage of the refined treatment plan and at least one aspect of the original treatment plan.

10. The method of claim 9, wherein a digital 3D representation of a planned arrangement of the teeth in the dental arch is provided for each stage of the refined treatment plan.

11. The method of claim 10, wherein the digital 3D representation of a planned arrangement is provided for at least each stage of the original treatment plan that occurred prior to the refinement request.

12. The method of claim 10, wherein the treatment timeline includes a visible separation between the one or more stages of the original treatment plan and the one or more stages of the refined treatment plan.

13. The method of claim 1, wherein the digital representation of at least a portion of the original treatment plan and the refined treatment plan includes model overlays between digital 3D representations of teeth arrangements in a first user selected phase of treatment and a second user selected phase of treatment.

14. The method of claim 1, wherein the providing step comprises providing a combination of aligners and brackets.

15. The method of claim 1, wherein the method further includes providing an original orthodontic treatment plan, wherein the providing includes the steps of:
receiving a digital representation of the patient's malocclusion; and
creating a target final arrangement of teeth to be achieved at the end of the treatment plan.

16. The method of claim 15, wherein creating the refined treatment plan includes modifying the target final arrangement of teeth.

17. (canceled)

18. A system for reviewing an orthodontic treatment path for a patient's dental arch having a current arrangement of teeth, comprising a computing device for executing the method of claim 1.

19. A non-transient, computer-readable medium containing program instructions for causing a computer to:
receive, from a remote site, a request for revision after at least one stage of the treatment plan has occurred or the dental arch has reached an arrangement of teeth different than the current arrangement, the revision including a modification to at least one aspect of an original treatment plan;
display, on a screen, a digital representation of at least a portion of the original treatment plan and a revised treatment plan for user approval, the revised treatment plan including one or more stages of treatment, wherein each stage includes a planned arrangement of teeth in the dental arch and at least one stage includes a modified arrangement of teeth as compared to the original treatment plan; and
transmit the revised treatment plan for fabrication of one or more orthodontic appliances after the user has approved the revised treatment plan displayed on the screen.

20. The non-transient, computer-readable medium of claim 19, wherein the program instructions are further configured to display a treatment timeline including a representation of each stage of the refined treatment plan and at least one stage of the original treatment plan.

21. The non-transient, computer-readable medium of claim 19, wherein the digital 3D representation of a planned arrangement is provided for each stage of the refined treatment plan, and wherein the digital 3D representation of a planned arrangement is provided for at least each stage of the original treatment plan that occurred prior to the refinement request.

22. The non-transient, computer-readable medium of claim 19, wherein the treatment timeline includes a visible separation between the stages of the original treatment plan and the stages of the refined treatment plan.
